(19) United States
(12) Patent Application Publication
(10) Pub. No.: US 2012/0116348 A1
(43) Pub. Date: May 10, 2012

(54) LIQUID MEDICINE INJECTION AMOUNT ADJUSTING METHOD, LIQUID MEDICINE INJECTION AMOUNT ADJUSTING APPARATUS, AND LIQUID MEDICINE INJECTING SYSTEM

(76) Inventors: Seiichi Katoh, Miyagi (JP); Yasuhiro Kawamura, Tokyo (JP)

(21) Appl. No.: 13/057,889

(22) PCT Filed: Aug. 4, 2009
(86) PCT No.: PCT/JP2009/064085

§ 371 Date: Jun. 1, 2011

(30) Foreign Application Priority Data
Aug. 8, 2008 (JP) 2008-205180
Jun. 30, 2009 (JP) 2009-154506

(51) Int. Cl. A61M 5/168

(52) U.S. Cl. 604/506; 604/67

(57) ABSTRACT

In a liquid medicine injecting system, an open route is formed in which a liquid medicine flows from a container that contains the liquid medicine to a blood vessel of a biological body via a micro-pump, a flow volume sensor, and a tube. A back pressure from the biological body directly operates against the micro-pump via the open route. When the flow volume of the liquid medicine is constant, power of the micro-pump has a constant relationship with the back pressure. The flow volume of the liquid medicine is adjusted to be a target volume by controlling the power of the micro-pump, and the power of the micro-motor is monitored. An abnormal injecting state of the liquid medicine caused by such as pulling out of an injection needle from the biological body is immediately detected with high accuracy based on the monitored result of the power of the micro-pump.

### TECHNICAL FIELD

The present invention generally relates to a liquid medicine injection amount adjusting method, a liquid medicine injection amount adjusting apparatus, and a liquid medicine injecting system using the liquid medicine injection amount adjusting apparatus in which an amount of a liquid medicine to be injected into a biological body from a liquid medicine container is adjusted.

### BACKGROUND ART

When a liquid medicine is injected into a biological body, an infusion apparatus has been generally used. In the infusion apparatus, one end of a tube is connected to a container containing a liquid medicine, and the liquid medicine is injected into the biological body via an injection needle connected to the other end of the tube. A liquid medicine injection amount adjusting apparatus is positioned in the middle of the tube for adjusting injection speed of the liquid medicine. Conventionally, the liquid medicine injection amount adjusting apparatus provides an infusion tube and a clamp, and a healthcare worker, for example, a nurse operates the clamp while watching a liquid medicine dripping state in the infusion tube.

In addition, a device called a liquid medicine injecting pump has been used. The liquid medicine injecting pump drives an injection tube by a motor having a mechanism which controls rotational speed of the pump. Alternatively, the liquid medicine injecting pump uses an ironing pump which presses the injection tube at a constant pressure. With this, the injecting speed (an injection amount of the liquid medicine per unit time) is adjusted.

When the injecting speed is adjusted in the conventional infusion apparatus, for example, a nurse visually confirms the size of the droplet in the infusion tube and the number of the droplets per unit time. Consequently, the adjustment of the injecting speed largely depends on personal experience and intuition. That is, it is difficult for a person having little experience to adjust the injecting speed to be an optimum value.

For example, the size of the droplet of the liquid medicine is largely affected by the viscosity, the concentration, and the surface tension of the liquid medicine. In addition, the viscosity and the surface tension are largely affected by temperature. That is, the size of the droplet is affected by the temperature, and it is difficult for the nurse to accurately estimate the size of the droplet by visual confirmation. When the temperature is changed during the (drip) infusion, the injecting speed is also changed. Consequently, the injecting speed must be always adjusted by the operation of the clamp. Similar to the above, in the liquid medicine injecting pump, the viscosity, the concentration, and the surface tension of the liquid medicine are changed by the kind of the medicine and the temperature; therefore, it is very difficult for the nurse (person) to determine the initial injecting speed and to maintain the injecting speed to be a constant value.

In order to solve the above problem, an apparatus has been proposed. In the apparatus, a liquid medicine to be injected into a biological body is contained in a container, the container is supported by a weight detecting mechanism, and the remaining weight of the liquid medicine is measured with the passage of time. Then the liquid medicine flowing out speed from the container is controlled with the passage of time based on the measured results so that a predetermined amount of the liquid medicine is injected within a predetermined period (for example, see Patent Document 1).

However, in the apparatus disclosed in Patent Document 1, when the following case occurs, the liquid medicine cannot be accurately injected into the biological body. That is, when the injection needle is dropped out of the biological body, or a part of a liquid medicine flowing route is separated from a normal route; a large amount of the liquid medicine flows out without being injected into the biological body.

For example, in a case where a liquid medicine is injected into a blood vessel of a biological body, when the posture of the biological body is changed, a tip of the injection needle may drop out of the blood vessel by being pulled, and the tip of the injection needle remains in tissue surrounding the blood vessel, and the liquid medicine is injected into the tissue. In some cases, the liquid medicine may be harmful for the tissue.

In addition, the blood vessel may be pressed by the injected liquid medicine in the tissue, and may be injured. Further, the blood flow is stopped by the pressure, and cells and tissue at the downstream side of the flow may necrotize. It is well known that there is a high possibility of the above phenomenon occurrence when the flowing amount of the liquid medicine is more than 50 to 100 ml/h.

In addition, a conventional liquid medicine injecting pump, in order to detect an abnormal state, for example, dropping out of an injection needle from a syringe, an individual sensor is utilized to detect the abnormal injecting state (for example, see Patent Document 2). However, in Patent Document 2, it is difficult to immediately detect the abnormal injecting state.
[Patent Document 1] Japanese Unexamined Patent Publication No. S63-212371
[Patent Document 2] Japanese Unexamined Patent Publication No. 2008-086581

### SUMMARY OF INVENTION

In an embodiment of the present invention, there is provided a liquid medicine injection amount adjusting method, a liquid medicine injection amount adjusting apparatus, and a liquid medicine injecting system using the liquid medicine injection amount adjusting apparatus in which an abnormal injecting state of a liquid medicine into a biological body is automatically detected with high accuracy and certainty, and a continuation of the injection of the liquid medicine into the biological body can be prevented in the abnormal injecting state.

To achieve one or more of these and other advantages, according to one aspect of the present invention, there is provided a liquid medicine injection amount adjusting method which adjusts an injection amount of a liquid medicine to be injected into a biological body from a container which contains the liquid medicine. The liquid medicine injection amount adjusting method includes a first step which controls power of a pump connected in the middle of a liquid medicine injecting tube route formed from the container to the biological body so that a flow volume of the liquid medicine flowing in the liquid medicine injecting tube route is maintained to be a target flow volume based on measured information of the flow volume of the liquid medicine flowing in the liquid medicine injecting tube route, and a second step which monitors the power of the pump in parallel with the first step.

### EFFECT OF INVENTION

According to an embodiment of the present invention, an abnormal injecting state of a liquid medicine into a biological body is automatically detected with high accuracy and certainty, and a continuation of the injection of the liquid medicine into the biological body can be prevented in the abnormal injecting state.

### BRIEF DESCRIPTION OF DRAWINGS

Features and advantages of the present invention will become more apparent from the following detailed description when read in conjunction with the accompanying drawings, in which:

FIG. 1 is a diagram showing a structure of a liquid medicine injecting system according to an embodiment of the present invention;

FIG. 2A is a cut-away side view of a micro-pump shown in FIG. 1;

FIG. 2B is a cross-sectional view of the micro-pump along line B-B of FIG. 2A;

FIG. 3A is a schematic diagram showing an operating principle of the micro-pump shown in FIG. 1;

FIG. 3B is another schematic diagram showing the operating principle of the micro-pump shown in FIG. 1;

FIG. 4A is a schematic diagram showing a flow volume sensor shown in FIG. 1;

FIG. 4B is a graph showing temperature distributions of a liquid medicine measured by the flow volume sensor shown in FIG. 1;

FIG. 5 is a flowchart showing processes of a process algorithm of a control unit when the liquid medicine is injected into a blood vessel of a biological body shown in FIG. 1;

FIG. 6 is a flowchart showing processes of an interruption process for determining an injecting state of the liquid medicine by the control unit shown in FIG. 1;

FIG. 7A is a diagram showing a state in which an injection needle has been normally inserted into the blood vessel of the biological body shown in FIG. 1;

FIG. 7B is a diagram showing a state in which the injection needle has been pulled out of the blood vessel of the biological body shown in FIG. 1;

FIG. 7C is a diagram showing a state in which a pool of the liquid medicine in the biological body shown in FIG. 1 has been expanded;

FIG. 8A is a diagram showing monitored results of power of the micro-pump when the liquid medicine has been normally injected into the biological body shown in FIG. 1;

FIG. 8B is a diagram showing monitored results of power of the micro-pump when the injection needle shown in FIG. 1 has been pulled out of the blood vessel and the liquid medicine has been injected to outside the blood vessel;

FIG. 8C is another diagram showing monitored results of the power of the micro-pump when the injection needle shown in FIG. 1 has been pulled out of the blood vessel and the liquid medicine has been injected to outside the blood vessel; and

FIG. 9 is a diagram showing a structure of a liquid medicine injecting system according to a modified example of the embodiment of the present invention.

### MODE(S) FOR CARRYING OUT THE INVENTION

Referring to the drawings, an embodiment of the present invention is described in detail.

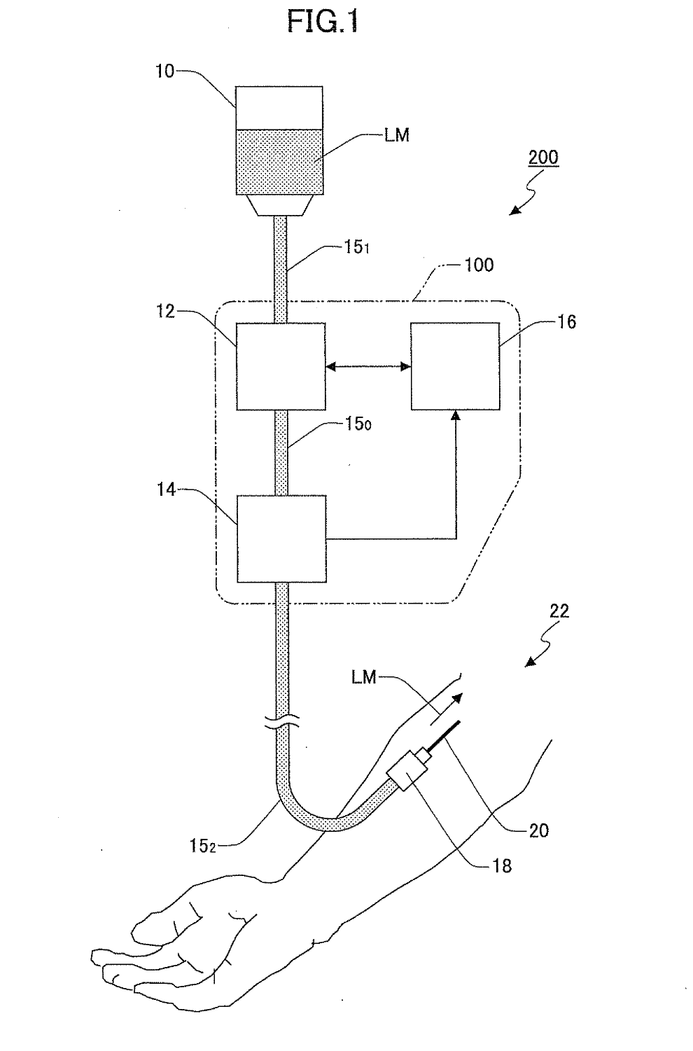

FIG. 1 is a diagram showing a structure of a liquid medicine injecting system 200 according to the embodiment of the present invention.

As shown in FIG. 1, the liquid medicine injecting system 200 includes a container 10 for containing a liquid medicine LM to be injected into a biological body 22, a liquid medicine injecting tube route, a liquid medicine injection amount adjusting apparatus 100, an attachment 18, and an injection needle 20. The liquid medicine injecting tube route includes tubes 15₁, 15₀, and 15₂. One end of the tube 15₁ is connected to the container 10 and the other end of the tube 15₁ is connected to the liquid medicine injection amount adjusting apparatus 100. One end of the tube 15₂ is connected to the liquid medicine injection amount adjusting apparatus 100 and the other end of the tube 15₂ is connected to the injection needle 20 via the attachment 18 which connects the injection needle 20 to the tube 15₂. The tube 15₀ is positioned in the liquid medicine injection amount adjusting apparatus 100. That is, the liquid medicine injection amount adjusting apparatus 100 is positioned in the middle of the liquid medicine injecting tube route.

As shown in FIG. 1, the liquid medicine injection amount adjusting apparatus 100 includes a micro-pump 12, a flow volume sensor 14, and a control unit 16. Elements in the liquid medicine injection amount adjusting apparatus 100 are described below in detail.

When the liquid medicine LM is injected into a part of the biological body 22, for example, a blood vessel, the container 10 is connected to the micro-pump 12 of the liquid medicine injection amount adjusting apparatus 100 via the tube 15₁. The tube 15₁ is a flexible tube formed of an individually expandable material having high elasticity.

The flow volume sensor 14 of the liquid medicine injection amount adjusting apparatus 100 is connected to the attachment 18 via the tube 15₂ to whose tip the injection needle 20 is secured. When the liquid medicine LM is injected into a blood vessel, for example, a nurse pricks the injection needle 20 into the biological body 22 via a body surface and adjusts the tip of the injection needle 20 inside the blood vessel. At this time, the end of the injection needle 20 or the attachment 18 is secured onto a body surface of the biological body 22 by using, for example, an adhesive tape so that the tip of the injection needle 20 is not pulled out of the blood vessel. In FIG. 1, the injection needle 20 has been secured to the biological body 22.

Similar to the tube 15₁, the tube 15₂ is a flexible tube, and even if the tip of the tube 15₂ is moved due to bending of the tube 15₂, the flow route of the liquid medicine LM can be obtained.

In the liquid medicine injecting system 200, from the container 10 to the blood vessel of the biological body 22, the flow route of the liquid medicine LM is formed by the tube 15₁, the liquid medicine injection amount adjusting apparatus 100, the tube 15₂, and the injection needle 20. In the middle of the flow route of the liquid medicine LM, a member which closes the flow route does not exist. That is, the flow route is an open route from the container 10 to the blood vessel of the biological body 22.

A valve to prevent a reverse flow of the liquid medicine LM can be positioned in the middle of the flow route from the container 10 to the injection needle 20. However, when the valve is positioned, a resistance force against the normal flow of the liquid medicine LM from the container 10 to the injection needle 20 is required not to influence the flow of the liquid medicine LM or must be negligibly small.

Next, a structure and functions of the liquid medicine injection amount adjusting apparatus 100 are described in detail.

The control unit 16 is electrically connected to the micro-pump 12 and the flow volume sensor 14.

As described above, the micro-pump 12 is connected to the flow volume sensor 14 via the tube 15₀. The material and the shape of the tube 15₀ are not particularly limited, when the tube 15₀ can connect the micro-pump 12 to the flow volume sensor 14 and the liquid medicine LM can flow in the tube 15₀.

In the embodiment of the present invention, as the micro-pump 12, a diaphragm pump is used in which a driving source is obtained from a piezoelectric element. The diaphragm pump is a kind of volume pumps and is manufacture by an MEMS (micro electro mechanical systems) technology.

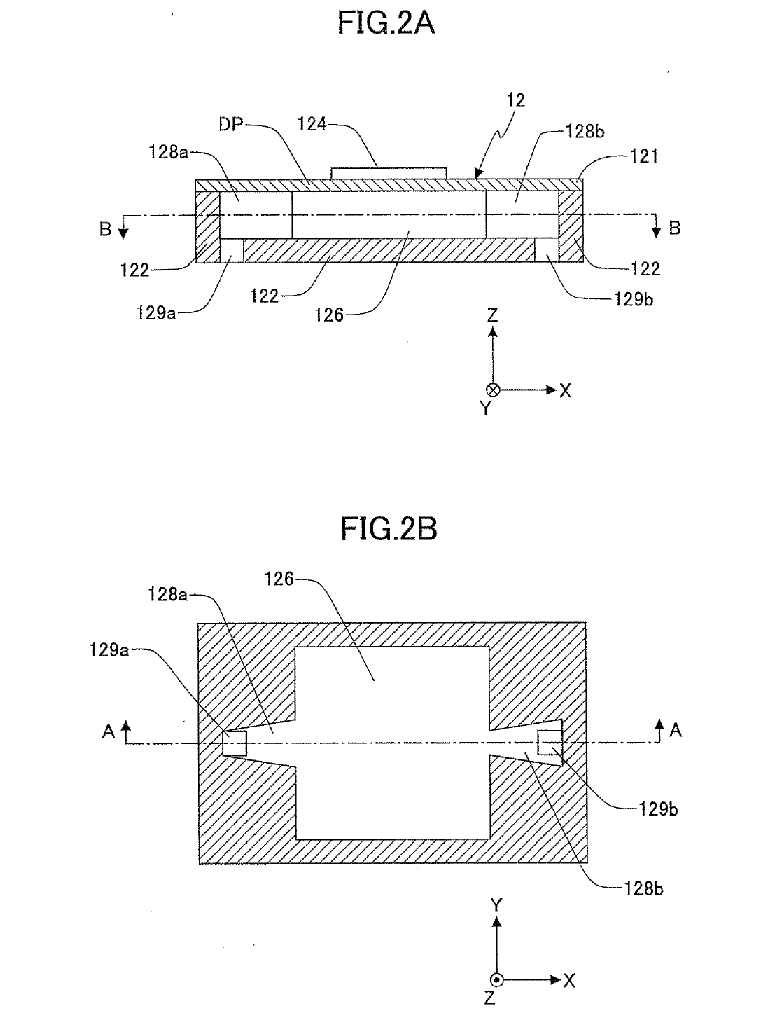

FIG. 2A is a cut-away side view of the micro-pump 12, and FIG. 2B is a cross-sectional view of the micro-pump 12 along line B-B of FIG. 2A. FIG. 2A corresponds to a cross-sectional view of the micro-pump 12 along line A-A of FIG. 2B.

As shown in FIG. 2A, the micro-pump 12 includes a first substrate 121 having a plate shape a part of which functions as a diaphragm, a second substrate 122 jointed to one surface (−Z side surface) of the first substrate 121, and a piezoelectric element 124 secured at a center part of the other surface (+Z side surface) of the first substrate 121. As an example, the first substrate 121 is formed of boronsilicate glass, and the second substrate 122 is formed of silicon. A part of the first substrate 121, including a part in contact with the piezoelectric element 124, is called a diaphragm part DP which functions as the diaphragm.

As shown in FIGS. 2A and 2B, a concave section is formed in the second substrate 122 from the surface facing the first substrate 121 by having a predetermined depth. The concave section includes a pressure chamber 126 having a rectangular shape in planar view positioned at a center part in the X and Y axes directions, a groove 128a connected to an end part of the pressure chamber 126 in the −X direction, and a groove 128b connected to another end part of the pressure chamber 126 in the +X direction. Actually, the pressure chamber 126 is formed when the first substrate 121 is jointed to the second substrate 122 so that the first substrate 121 covers the concave section formed in the second substrate 122. However, for the sake of simplicity, it is described that the pressure chamber 126 is formed in the second substrate 122.

A through hole 129a, which connects an internal space of the groove 128a to the outside of the second substrate 122, is formed in a bottom wall of the second substrate 122 corresponding to the −X end part in the groove 128a. In addition, a through hole 129b, which connects an internal space of the groove 128b to the outside of the second substrate 122, is formed in a bottom wall of the second substrate 122 corresponding to the +X end part in the groove 128b.

The through hole 129a functions as an inlet of the liquid medicine LM to the internal space of the micro-pump 12 including the pressure chamber 126, and the through hole 129b functions as an outlet of the liquid medicine LM from the internal space of the micro-pump 12. In the following, the through hole 129a is described as the inlet 129a, and the through hole 129b is described as the outlet 129b. The inlet 129a is connected to a tube member (not shown) which is a supplying opening of the liquid medicine LM to the micro-pump 12, and the outlet 129b is connected to another tube member (not shown) which is a discharging opening of the liquid medicine LM from the micro-pump 12.

As shown in FIG. 2B, the cross sectional area of each of the grooves 128a and 128b is gradually widened from the −X end to the +X end (from the inlet 129a to the outlet 129B), and also functions as a diffuser. In the following, the grooves 128a and 128b are described as the diffusers 128a and 128b. Generally, a diffuser converts a kinetic energy of a fluid into a pressure energy.

As described above, in the embodiment of the present invention, a flow route of the liquid medicine LM is formed from the inlet 129a to the outlet 129b in the second substrate 122 via the diffuser 128a, the pressure chamber 126, and the diffuser 128b. In the flow route, since a member to close the flow route does not exist, an open route connecting from the inlet 129a to the outlet 129b is formed. That is, the micro-pump 12 is a valve-less micro-pump.

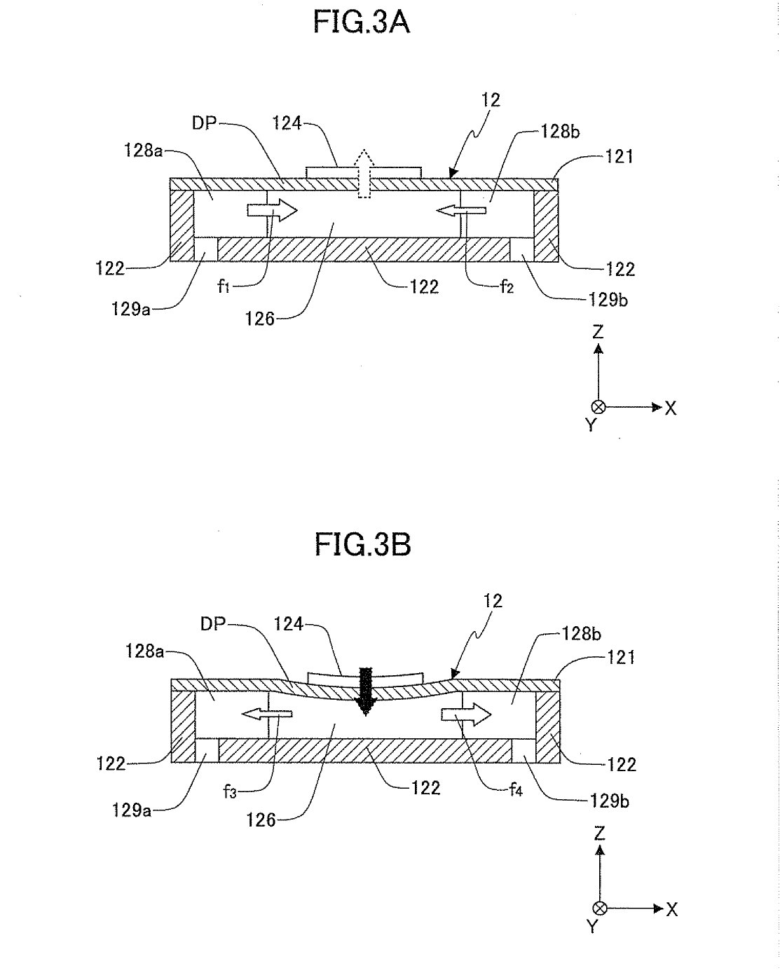

FIG. 3A is a schematic diagram showing an operating principle of the micro-pump 12, and FIG. 3B is another schematic diagram showing the operating principle of the micro-pump 12.

In the embodiment of the present invention, when a voltage has not been applied to the piezoelectric element 124, as shown in FIG. 3A, the diaphragm part DP of the first substrate 121 jointed to the piezoelectric element 124 maintains a flat surface without being bent (deflected). When a voltage has been applied to the piezoelectric element 124, as shown in FIG. 3B, the diaphragm part DP of the first substrate 121 is bent in the −Z direction as shown by the black arrow, and the pressure chamber 126 is contracted.

Therefore, when voltage pulses are applied to the piezoelectric element 124, the diaphragm part DP can be vibrated. That is, by applying the voltage pulses to the piezoelectric element 124, contraction and expansion (from the contraction) of the pressure chamber 126 are repeated.

The contraction rate of the pressure chamber 126 (the bending amount of the diaphragm part DP) is determined by the pulse amplitude V of the voltage pulse (or the product VH (pulse area) of the pulse amplitude V and the pulse width H). The number of the vibrations (the number of repetitions of the construction and the expansion) of the pressure chamber 126 is determined by the frequency ω (=1/T) (T is the pulse period) of the voltage pulses.

As shown in FIG. 3A, when the pressure chamber 126 is expanded (actually, the expansion rate is 1), the liquid medicine LM flows into the pressure chamber 126 from the inlet 129a and the outlet 129b. In FIG. 3A, the direction and the size of the liquid medicine LM flowing into the pressure chamber 126 from the inlet 129a is shown by the white arrow “f₁”, and the direction and the size of the liquid medicine LM flowing into the pressure chamber 126 from the outlet 129b is shown by the white arrow “f₂”.

The liquid medicine LM shown by the white arrow “f₁” passes through the diffuser 128a, and the liquid medicine LM shown by the white arrow “f₂” passes through the diffuser 128b. As described above, the cross sectional area of each of the diffusers 128a and 128b is gradually widened in the +X direction. Therefore, the diffusers 128a and 128b give a small resistance to a fluid (the liquid medicine LM) flowing in the +X direction and give a large resistance to the fluid flowing in the −X direction. Therefore, in FIG. 3A, since the fluid shown by the white arrow “f₁” receives the small resistance from the diffuser 128a, the flow volume of the fluid shown by the white arrow “f₁” is great, and since the fluid shown by the white arrow “f₂” receives the large resistance from the diffuser 128b, the flow volume of the fluid shown by the white arrow “f₂” is small.

On the other hand, as shown in FIG. 3B, when the pressure chamber 126 is contracted, the fluid (the liquid medicine LM) flows into the inlet 129a and the outlet 129b from the pressure chamber 126. The direction and the size of the fluid flowing into the inlet 129a from the pressure chamber 126 is shown by the white arrow “f₃”, and the direction and the size of the fluid flowing into the outlet 129b from the pressure chamber 126 is shown by the white arrow “f₄”. Since the fluid shown by the white arrow “f₃” receives the large resistance from the diffuser 128a, the flow volume of the fluid shown by the white arrow “f₃” is small, and since the fluid shown by the white arrow “f₄” receives the small resistance from the diffuser 128b, the flow volume of the fluid shown by the white arrow “f₄” is great.

When the pressure chamber 126 is contracted and expanded once, a net volume of |f₁−f₃| of the fluid flows into the pressure chamber 126 from the inlet 129a, and a net volume of |f₄−f₂| of the fluid flows out from the pressure chamber 126 to the outlet 129b. That is, the net volume “f”=|f₁−f₃|=|f₄−f₂| of the fluid flows from the inlet 129a to the outlet 129b. The fluid is assumed to have a non-compression property. When the volume of the pressure chamber 126 is defined as W and the contraction rate of the pressure chamber 126 is defined as β, a relationship “f”=W (1−β) is obtained.

When the contraction and the expansion of the pressure chamber 126 are repeated, a constant flow of the fluid is generated from the inlet 129a to the outlet 129b. When the number of repetitions of the contraction and the expansion of the pressure chamber 126 per unit time is defined as ω (the frequency of the voltage pulse), a bulk flow volume per unit time F=ωf=ωW(1−β) of the fluid flows from the inlet 129a to the outlet 129b.

The bulk flow volume F can be controlled by adjusting at least one of the pulse amplitude V, the pulse width H (the pulse area VH), and the pulse period T (the frequency=1/T) of the voltage pulse to be applied to the piezoelectric element 124.

When the pulse amplitude V (the pulse area VH) of the voltage pulse to be applied to the piezoelectric element 124 is made to be large (small), the amount of the contraction and the expansion of the piezoelectric element 124; that is, the bending (deflection) amount of the diaphragm part DP becomes large (small). Therefore, when the pulse amplitude V (the pulse area VH) of the voltage pulse is changed, the contraction and expansion rate (1−β) of the pressure chamber 126 can be adjusted. With this, the bulk flow volume F=ωW(1−ω) can be controlled.

In addition, when the frequency of the voltage pulse is made to be large (small), the number of vibrations of the diaphragm part DP; that is, the number of repetitions of the contraction and the expansion of the pressure chamber 126 per unit time ω, becomes large (small). Therefore, when the frequency of the voltage pulse is changed, the number of repetitions of the contraction and the expansion of the pressure chamber 126 per unit time ω can be adjusted. With this, the bulk flow volume F=ωW(1−ω) can be controlled. The frequency of the voltage pulse is equal to the number of repetitions of the contraction and the expansion of the pressure chamber 126 per unit time ω, in principle; therefore, the frequency ω of the voltage pulse is used.

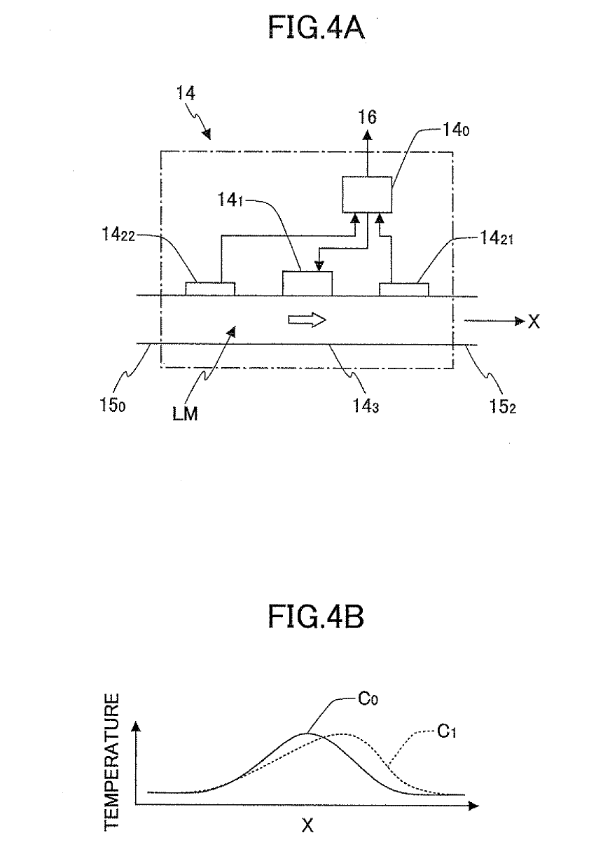

As the flow volume sensor 14, as an example, a thermal type mass flow volume sensor shown in FIG. 4A is used. FIG. 4A is a schematic diagram showing the thermal type mass flow volume sensor 14. FIG. 4B is a graph showing temperature distributions of the liquid medicine LM measured by the thermal type mass flow volume sensor 14.

As shown in FIG. 4A, the thermal type mass flow volume sensor 14 includes a main body 14₀, a tube route 14₃ in which the fluid flows, a heat source 14₁ positioned on the tube route 14₃, and a pair of temperature sensors 14₂₂ and 14₂₁ symmetrically positioned at the corresponding upstream and downstream sides by sandwiching the heat source 14₁.

In the thermal type mass flow volume sensor 14, while the liquid medicine LM is flowing in the tube route 14₃, heat is applied to the liquid medicine LM in the tube route 14₃ by using the heat source 14₁, and the temperature sensors 14₂₁ and 14₂₂ measure heat amounts transmitted from the liquid medicine LM via the tube walls of the tube route 14₃. The measured results of the temperature sensors 14₂₁ and 14₂₂ are sent to the main body 14₀.

The main body 14₀ of the thermal type mass flow volume sensor 14 obtains the flow volume of the liquid medicine LM based on the measured results (measured information) of the temperature sensors 14₂₁ and 14₂₂. When the liquid medicine LM is not flowing (stays) in the tube route 14₃, since the heat from the heat source 14₁ is uniformly transmitted to the liquid medicine LM, the temperature distribution of the liquid medicine LM in the tube route 14₃ shows a symmetrical mount-like shape with the positioned position of the heat source 14₁ as the center as shown in C₀ of FIG. 4B. In this case, the measured results of the temperature sensors 14₂₁ and 14₂₂ are the same, and the difference between the measured results is 0.

On the other hand, when the liquid medicine LM is flowing in the +X direction shown by the white arrow of FIG. 4A, the temperature distribution of the liquid medicine LM in the tube route 14₃ shows an asymmetrical mount-like shape whose peak is shifted in the +X direction as shown in C₁ of FIG. 4B. In this case, the measured result of the temperature sensor 14₂₁ becomes greater than the measured result of the temperature sensor 14₂₂, and the difference between the measured results becomes a positive value when the measured result of the temperature sensor 14₂₂ is determined to be the reference. Based on the above principle, the thermal type mass flow volume sensor 14 (the main body 14₀) obtains the flow volume (including the flowing direction) of the liquid medicine LM flowing in the tube route 14₃ from the difference between the measured results.

When the thermal type mass flow volume sensor 14 is used, the flow volume can be measured at a high speed because of the principle of the sensor. In addition, since a probe is not required to insert into the fluid, the flow volume can be accurately measured without disturbing the flow of the fluid.

The control unit 16 includes, for example, a microcomputer as a central element, and controls all elements in the liquid medicine injection amount adjusting apparatus 100.

As described above, the control unit 16 is electrically connected to the micro-pump 12 and the flow volume sensor 14. The measured result of the flow volume of the liquid medicine LM is supplied to the control unit 16 from the flow volume sensor 14.

The control unit 16 adjusts the voltage pulse to be applied to the piezoelectric element 124 of the micro-pump 12 based on the measured result of the flow volume so that the flow volume of the liquid medicine LM becomes a predetermined target volume. Specifically, the control unit 16 adjusts at least one of the pulse amplitude V, the pulse area VH, and the frequency ω (=1/T) of the voltage pulse. That is, the control unit 16, the micro-pump 12, and the flow volume sensor 14 form a feedback control system which controls the flow volume of the liquid medicine LM (power of the micro-pump 12) by feedback control. The control of the micro-pump 12 is described below in detail.

At least one of the connection between the control unit 16 and the micro-pump 12, and the connection between the control unit 16 and the flow volume sensor 14 can be formed of radio communications. In addition, as the feedback control, so-called PID control (proportional control, integral control, and derivative control) can be used. When the PID control is used, the control unit 16 can be formed of an analog circuit of an operational amplifier.

The control unit 16 also monitors the power of the micro-pump 12. The power of the micro-pump 12 is pressure (energy) to be applied to the fluid (the liquid medicine LM) so that the fluid flows in the forward direction. However, as the power, it is not necessary to consider specific pressure (energy) to be applied to the fluid from the micro-pump 12, but it is sufficient to consider an amount of the pressure. From the structure of the micro-pump 12, the power becomes a function P(V, ω) or P(VH, ω) of the pulse amplitude V, or the pulse area VH, and the frequency ω (=1/T) of the voltage pulse. However, the power P must be approximated to the pressure, or must be proportional to the pressure in good approximation.

For example, the product of the pulse amplitude V, (or the pulse area VH), and the frequency ω (=1/T) of the voltage pulse is defined as the power P. That is, P(V, ω)≡Vω or P(VH, ω)≡VHω can be defined. When the pulse amplitude V (or the pulse area VH) is always constant V₀ (or VH₀), and the frequency ω is only variable, P(V₀, ω)≡ω or P(VH₀, ω)≡ω can be simply defined.

In addition, when the frequency ω is always constant ω₀, and the pulse amplitude V (or the pulse area VH) is variable, P(V, ω₀)≡V or P(VH, ω₀)≡VH can be simply defined. When the above conditions are not satisfied, a relationship between the power P and the pressure has been obtained beforehand, and the power P is converted into the pressure by using the relationship.

The control unit 16 includes a storage unit (not shown) and stores the monitored results (monitored information) of the power P in the storage unit at each predetermined time interval (Δt). The stored monitored results are erased when a predetermined period has passed after storing the monitored result. Therefore, the newest monitored results “n” (a constant number) within the predetermined period have been stored in the storage unit.

The control unit 16 determines (diagnoses) the injecting state of the liquid medicine LM based on the monitored results of the power P of the micro-pump 12. The determining method is described below in detail. When the control unit 16 detects an abnormal injecting state of the liquid medicine LM, the control unit 16 stops the injection of the liquid medicine LM, and performs an emergency procedure, for example, a procedure to give a warning. When the injection of the predetermined amount (the target amount) of the liquid medicine LM has been normally completed, the control unit 16 performs a completion procedure, for example, a procedure to stop the injection of the liquid medicine LM.

In addition, the control unit 16 further includes interfaces such as an operating panel (not shown) on which an operator (nurse) inputs a target injection amount of the liquid medicine LM, an injection period of the liquid medicine LM, and so on; a display panel on which the injecting state of the liquid medicine LM is displayed, and a warning device for informing an abnormal injecting state of the liquid medicine LM.

Next, in the liquid medicine injection amount adjusting apparatus 100 of the embodiment of the present invention, an injecting method of the liquid medicine LM into a blood vessel of the biological body 22, and an abnormal injecting state detecting method are described by using an example when the injection needle 20 is pulled out of a blood vessel of the biological body 22, with the principles of the methods.

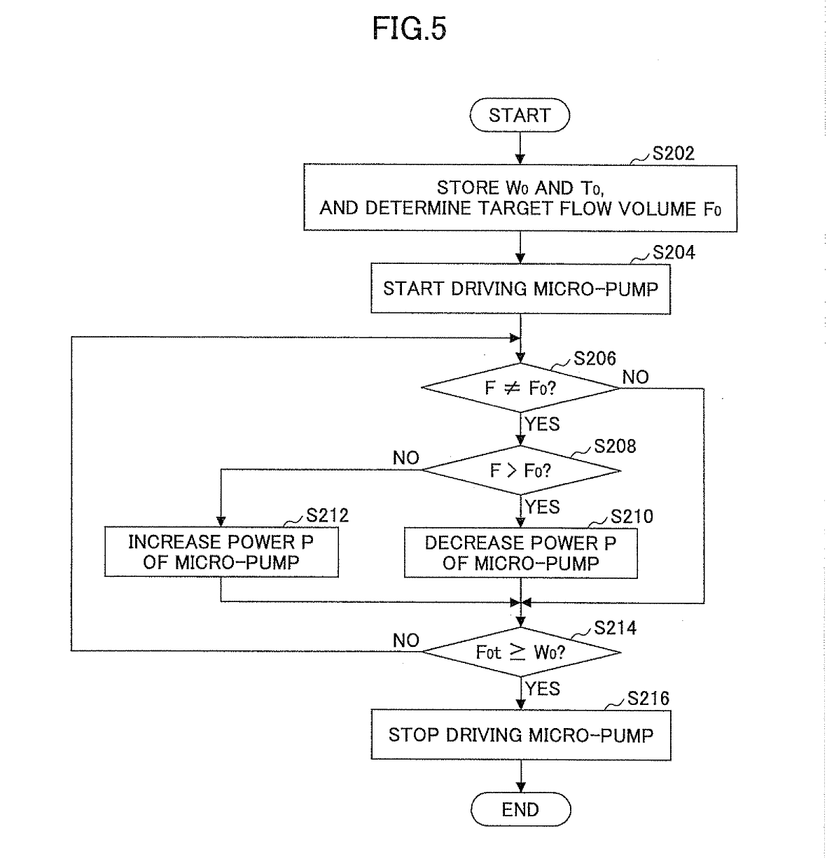

FIG. 5 is a flowchart showing processes corresponding to a process algorithm of the control unit 16 when the liquid medicine LM is injected into a blood vessel of the biological body 22. Specifically, in FIG. 5, the processes are performed by the CPU in the control unit 16.

In FIG. 5, before starting an injection of the liquid medicine LM into a blood vessel of the biological body 22, an operator inputs a total amount (a target injection amount) W₀ of the liquid medicine LM to be injected into the blood vessel of the biological body 22, the injection completion target time T₀ when the total amount W₀ is to be completely injected into the blood vessel of the biological body 22, and an instruction to start the injection of the liquid medicine LM on the operating panel.

The control unit 16 stores the target injection amount W₀ and the injection completion target time T₀ in the storage unit, and determines a target flow volume per unit time T₀ of the liquid medicine LM (S202).

Then the control unit starts driving the micro-pump 12 (S204).

Next, in processes of S206 through S212, the control unit 16 adjusts the power P of the micro-pump 12 so that the flow volume F becomes equal to the target flow volume F₀, based on a comparison result between the flow volume F of the liquid medicine LM reported from the flow volume sensor 14 and the target flow volume T₀.

That is, the control unit 16 determines whether the flow volume F is not equal to the target flow volume F₀ (F≠F₀?) (S206). When the flow volume F is not equal to the target flow volume F₀ (YES in S206), the control unit 16 determines whether the flow volume F is greater than the target flow volume F₀ (F>F₀?) (S208). When the flow volume F is greater than the target flow volume F₀ (YES in S208), the control unit 16 decreases the power P of the micro-pump 12 (S210). When the flow volume F is smaller than the target flow volume F₀ (NO in S208), the control unit 16 increases the power P of the micro-pump 12 (S212).

In order to adjust the flow volume F, the control unit 16 adjusts the pulse amplitude V (the pulse area VH) of the voltage pulse while maintaining the frequency ω of the voltage pulse to be constant, adjusts the frequency ω of the voltage pulse while maintaining the pulse amplitude V (the pulse area VH) of the voltage pulse to be constant, or adjusts both of the pulse amplitude V (the pulse area VH) and the frequency ω of the voltage pulse.

When the flow volume F is equal to the target flow volume F₀ (NO in S206), or after the processes in S210 and S212, the control unit 16 compares a injected amount F₀t at the time “t” with the target injection amount W₀ of the liquid medicine LM (S214). The time “t” is elapsed time after starting the injection of the liquid medicine LM.

When the injected amount F₀t is less than the target injection amount W₀ of the liquid medicine LM (F₀t<W₀) (NO in S214), the process returns to S206, and the processes from S206 through S214 are repeated. When the injected amount F₀t is the target injection amount W₀ or more of the liquid medicine LM (YES in S214), the control unit 16 determines that the liquid medicine LM has been normally injected in the blood vessel of the biological body 22, and stops driving the micro-pump 12 (S216). In addition, the control unit 16 performs a completion process such as a reporting process of the completion of the injection of the liquid medicine LM. With this, a series routine process of the injection of the liquid medicine LM into the blood vessel of the biological body 22 is completed.

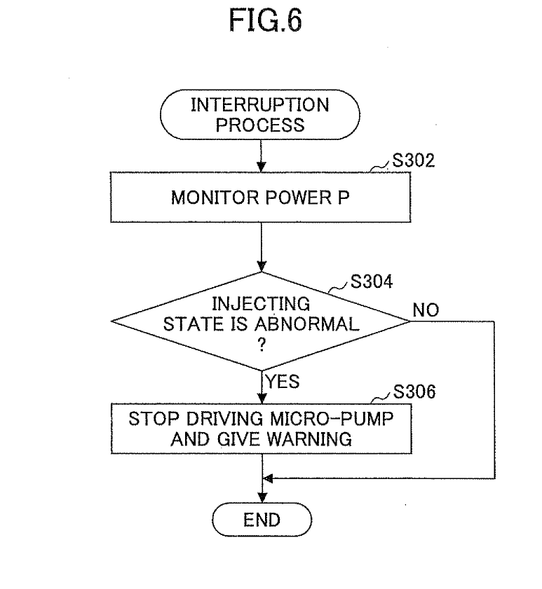

In the embodiment of the present invention, during the processes of the injection of the liquid medicine LM into the blood vessel of the biological body 22, the control unit 16 monitors the power P of the micro-pump 12, and determines (diagnoses) the injecting state of the liquid medicine LM based on the monitored result of the power P of the micro-pump 12. The control unit 16 determines the injecting state of the liquid medicine LM by using an interruption process (routine) shown in FIG. 6. After describing the determining principle, the interruption process shown in FIG. 6 is described.

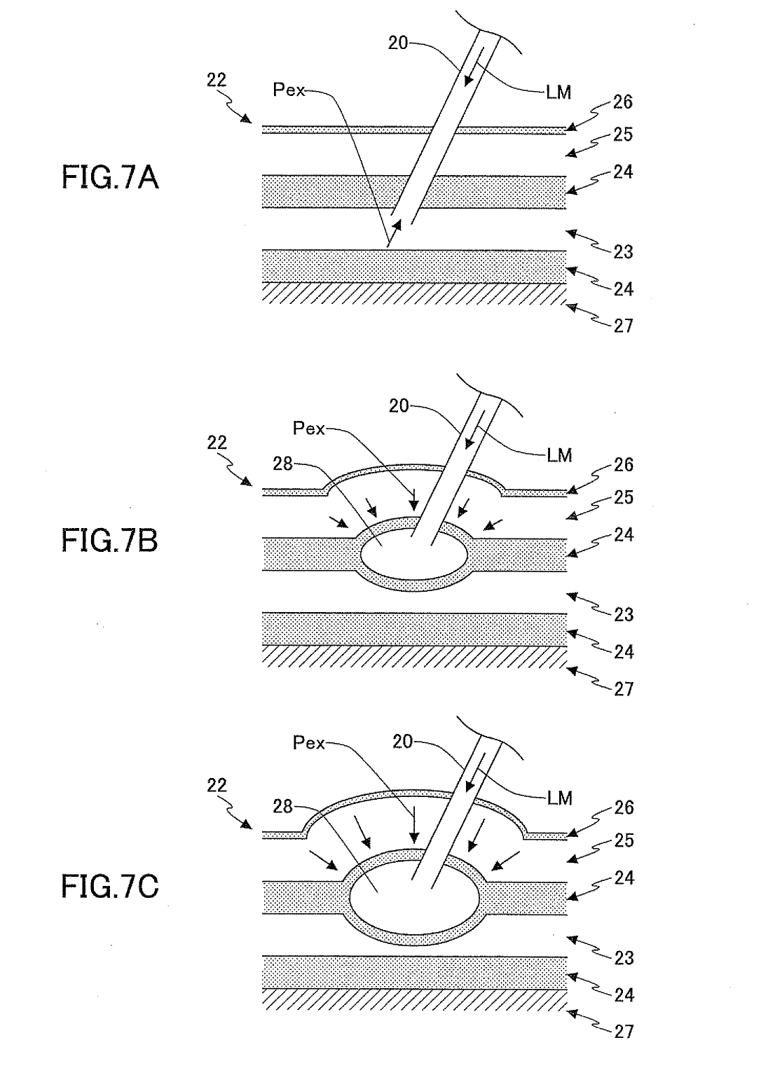

FIG. 7A is a diagram showing a state in which the injection needle 20 has been normally inserted into the blood vessel of the biological body 22.

In FIG. 7A, the tip of the injection needle 20 has been inserted into a blood vessel 23 via an epidermis 26, a dermis 25, and a hypodermal tissue 24. In FIG. 7A, a muscle 27 is also shown.

As described above, in the liquid medicine injecting system 200 of the embodiment of the present invention, one open route is formed from the container 10 to the blood vessel 23 of the biological body 22. In a normal injecting state, since the tip of the injection needle 20 stays in the blood vessel 23, a back pressure Pex equal to a pulse pressure operates against the liquid medicine LM from the blood vessel 23. Therefore, the control unit 16 adjusts the pulse amplitude V (or the pulse area VH) and/or the frequency ω of the voltage pulse to be applied to the micro-pump 12 by the flow volume control processes from S206 through S212 shown in FIG. 5 so that the power P of the micro-pump 12 to be applied to the liquid medicine LM becomes more than the back pressure Pex (P>Pex), and adjusts the flow volume F of the liquid medicine LM to be the target flow volume F₀.

In more detail, a viscosity resistance Pvr from the tube 15₂ and the wall of the injection needle 20 operates against the liquid medicine LM. Therefore, the control unit 16 adjusts the pulse amplitude V (or the pulse area VH) and/or the frequency ω of the voltage pulse to be applied to the micro-pump 12 so that P=Pex+Pvr, and adjusts the flow volume F of the liquid medicine LM to be the target flow volume F₀.

The back pressure Pex from the blood vessel 23 is not always constant and can be changed due to a change of a posture (for example, a standing posture or a sleeping posture) of the biological body 22. In addition, the viscosity of the liquid medicine LM generally depends on temperature, and the viscosity resistance Pvr is changed by a change of ambient temperature. However, by the flow volume control processes from S206 through S212 shown in FIG. 5, the flow volume F of the liquid medicine LM is always adjusted to the target flow volume F₀.

The liquid medicine injection amount adjusting apparatus 100 of the present embodiment functions as a current source in an analogy with an electric circuit. As it is understandable from the analogy, the power P of the micro-pump 12 has a constant relationship with the back pressure Pex when the flow volume F is maintained to be the target flow volume F₀. Therefore, when the power P is monitored while adjusting the flow volume F of the liquid medicine LM to the target flow volume F₀, a change of the back pressure Pex is obtained and the injecting state of the liquid medicine LM can be obtained (diagnosed) from the change of the back pressure Pex.

Next, as an example of the injecting state of the liquid medicine LM, a case is described in which the injection needle 20 has been pulled out of the blood vessel 23. FIG. 7B is a diagram showing a state in which the injection needle 20 has been pulled out of the blood vessel 23 of the biological body 22.

As shown in FIG. 7B, the tip of the injection needle 20 has been pulled out of the blood vessel 23 and stays in the hypodermal tissue 24 surrounding the blood vessel 23 without being pulled out of the biological body 22. In this case, the liquid medicine LM is injected into the hypodermal tissue 24.

In this case, by the flow volume control processes from S206 through S212 shown in FIG. 5, the liquid medicine LM of the target flow volume F₀ always flows from the tip of the injection needle 20. Consequently, a pool 28 of the liquid medicine LM is formed in the hypodermal tissue 24, and the pool 28 is expanded with the passage of time. On the other hand, the back pressure Pex operates against the pool 28 of the liquid medicine LM in the hypodermal tissue 24 so as to prevent the pool 28 from being expanded (to stop the flow of the liquid medicine LM into the hypodermal tissue 24). As shown in FIG. 7C, it can be estimated that the back pressure Pex becomes great corresponding to the amount of the liquid medicine LM in the pool 28. FIG. 7C is a diagram showing a state in which the pool 28 of the liquid medicine LM has been expanded.

In order to solve the above problem, the control unit 16 monitors the power P of the micro-pump 12 by the interruption process shown in FIG. 6 while adjusting the flow volume F of the liquid medicine LM to the target flow volume F₀ by the flow volume control processes from S206 through S212 shown in FIG. 5. As described above, as the power P it is not necessary that the power P is specific power to be applied to the liquid medicine LM from the micro-pump 12. For example, the power P is defined as P=P(V, ω)≡Vω (or P=P(VH, ω)≡VHω.

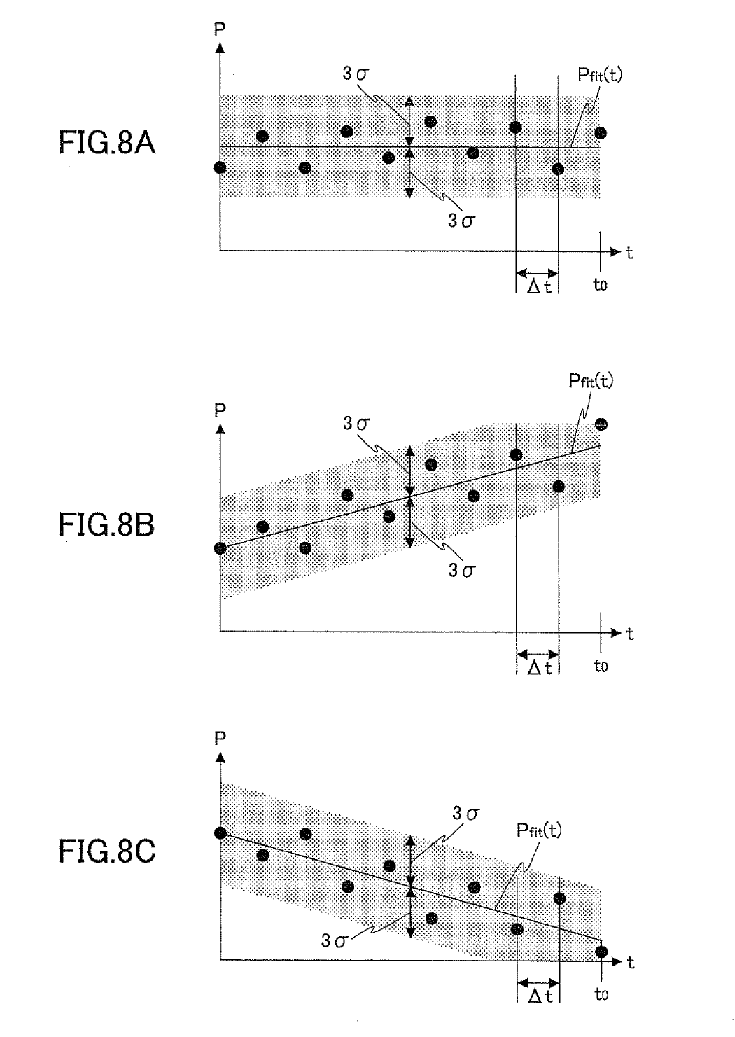

FIGS. 8A, 8B, and 8C show examples of monitored results of the power P of the micro-pump 12. As described above, the control unit 16 stores monitored results of the power P of the micro-pump 12 at each predetermined time interval Δt in the storage unit. The stored monitored results are erased when a predetermined period has passed after storing the monitored result. Therefore, the “n” (a constant number) newest monitored results within the predetermined period from the current time t₀ through (t₀−nΔt₀) have been stored in the storage unit. In FIGS. 8A, 8B, and 8C, it is determined that n=10 due to the space limitation of the paper of the drawings; however, the “n” can be arbitrarily determined corresponding to the requiring accuracy.

The control unit 16 obtains a time function P_{fit}(t) by applying a least square (fitting) method to the storing monitored results of the power P. In the time period nΔt, it is assumed that linear approximation of the power P in the time change can be sufficiently obtained. That is, the time period nΔt is selected so that the linear approximation of the power P in the time change is sufficiently obtained. As a result, it is given that P_{fit}(t)=a₀+a₁t. The coefficients a₀ and a₁ can be obtained by the least square method.

In FIG. 8A, the monitored results of the power P of the micro-pump 12 are shown when the liquid medicine LM has been normally injected. The monitored results of the power P are dispersed due to the time change of the back pressure Pex from the blood vessel 23. The dispersion is quantitatively defined as three times the standard deviation σ obtained from the least square method. The time change rate a₁ of the power P is negligibly small relative to the size of the dispersion 3σ. That is, |a₁nΔt|<<3σ. In this case, it is determined (diagnosed) that the liquid medicine LM is stably injecting into the blood vessel 23.

In FIG. 8B, the monitored results of the power P of the micro-pump 12 are shown when the injection needle 20 has been pulled out of the blood vessel 23 and the liquid medicine LM has been injected into the hypodermal tissue 24 as shown in FIGS. 7A and 7B. As described above, the back pressure Pex to be operated against the liquid medicine LM from the hypodermal tissue 24 becomes great when the amount of the liquid medicine LM in the pool 28 is increased. Consequently, as shown in FIG. 8B, the power P is increased with the passage of time. In this case, the time change rate a₁ of the power P cannot be negligible relative to the size of the dispersion 3σ. That is, when a₁nΔt>3σ, the control unit 16 determines that the injection needle 20 has been pulled out of the blood vessel 23 and the liquid medicine LM has been injected into outside the blood vessel 23.

When the tip of the injection needle 20 is pulled out of the biological body 22, the back pressure Pex to be operated against the liquid medicine LM becomes equal to the atmospheric pressure. In this case, as shown in FIG. 8C, the power P of the micro-pump 12 is attenuated with the passage of time. Therefore, when the time change rate a₁ of the power P satisfies a₁nΔt<−3σ, the control unit 16 determines that the injection needle 20 has been pulled out of the biological body 22.

Further, in addition to the change of the power P with the passage of time, the control unit 16 can determine whether the injecting state of the liquid medicine LM is a normal state from that the power P is within a normal state. However, since it can be assumed that the power P may be unstable, the control unit 16 monitors whether the function P_{fit}(t₀) is within the normal state.

Further, in addition to the pulling out of the injection needle 20 from the biological body 22, the power P may become unstable due to a breakage of the container 10, the tube 15₁, 15₂, or 15₀; a breakdown of the micro-pump 12 or the flow volume sensor 14; and so on. In this case, similar to the case shown in FIG. 8A, the power P becomes constant with the passage of time; however, it can be assumed that the size of the dispersion of the power P becomes great. Therefore, the control unit 16 determines that an abnormal state has occurred when the deviation σ has been more than a predetermined limit.

In addition, an abnormal state, which immediately recovers from the abnormal state, may temporarily occur in the liquid medicine injecting system 200 by the following reasons. That is, the reasons are the unstableness of the power source (the piezoelectric element 124) of the micro-pump 12, the unstableness of the feedback control, noise generated from measurement errors by the flow volume sensor 14, and a temporary change of the back pressure Pex caused by a change of the posture of the biological body 22. In order to exclude the temporarily abnormal state, the following four determining methods can be used.

In a first determining method, the control unit 16 stores a value of the time change rate a₁ (parameter) at each measurement time in the storage unit, averages the storing values of most recent “m” parameters a₁, and determines the injecting state of the liquid medicine LM by using the average value (a moving average value at each predetermined time interval Δt) with the use of the same methods shown in FIGS. 8A, 8B, and 8C.

In a second determining method, the control unit 16 obtains a value of the time change rate a₁ (parameter) of the power P by applying the least square method to the “n” monitored results of the power P monitored at each predetermined time interval Δtn and stores the obtained parameter a₁ in the storage unit. Then the control unit 16 averages the storing values of most recent “m” parameters a₁, and determines the injecting state of the liquid medicine LM by using the average value (a moving average value at each predetermined time interval Δtn) with the use of the same methods shown in FIGS. 8A, 8B, and 8C.

In a third determining method, the control unit 16 obtains a value of the time change rate a₁ (parameter) of the power P by applying the least square method to the “n” monitored results of the power P monitored at each predetermined time interval Δtn and stores the obtained parameter a₁ in the storage unit. Then the control unit 16 compares a value of the storing most recent parameters a₁ with a predetermined threshold value and determines the injecting state of the liquid medicine LM. In this case, when the control unit 16 detects an abnormal injecting state, the control unit 16 further averages the storing “m” most recent parameters a₁ and determines the injecting state of the liquid medicine LM by using the average value (a moving average value at each predetermined time interval Δtn). When the control unit 16 further detects an abnormal injecting state, the control unit 16 finally determines that an abnormal injecting state occurs.

In a fourth determining method, when the number of abnormal detection times is more than a predetermined number in the most recent “m” times of the determination (diagnosis), the control unit 16 determines that an abnormal state occurs.

In the first through fourth determining methods, the time interval Δt and the number of samples “n” and “m” are arbitrarily determined.

It is well known that the biological body 22 may be injured when the liquid medicine LM is injected into outside the blood vessel 23 for more than 30 minutes. Therefore, a total monitoring period Δtn or Δtm is determined to be 10 to 20 minutes. In the second through fourth determining methods, it is determined that, for example, Δt=1 second, n=60, and m=10. By using the first through fourth determining methods, the liquid medicine injection amount adjusting apparatus 100 can be stably operated without detecting a temporarily abnormal state by the averaging effect and the double determinations.

In addition, when the size of the temporary change of the parameter a₁ due to the above reasons has been known, a threshold value for the parameter a₁ can be determined in experience. When the parameter a₁ or the moving average of the parameters a₁ is more than the threshold value, it can be determined that an abnormal state occurs in the injection of the liquid medicine LM. Further, it is possible that a threshold value is determined for the parameter a₁, and another threshold value is determined for the moving average of the parameters a₁.

In addition, when it can be assumed that the power P is proportional to the back pressure Pex, or the power P is proportional to the back pressure Pex in a good approximation; the injecting state of the liquid medicine LM can be determined by using a parameter a₀ (an absolute value of the power P), with/without using the parameter a₁ (the time change rate of the power P). In this case, the threshold value is determined based on the deviation σ of the power P or in experience. When a value of the parameter a₀ or a moving average value of the parameters a₀ is more than the threshold value, it is determined that an abnormal injecting state occurs.

By using the interruption process shown in FIG. 6, the control unit 16 monitors the power P (S302), and determines whether the injecting state of the liquid medicine LM is abnormal based on one of the above principles and methods (S304). When it is determines that the injecting state of the liquid medicine LM is abnormal (YES in S304), the control unit 16 stops driving the micro-pump 12 (stops injecting the liquid medicine LM), and gives a warning (S306). With this, an emergency procedure is completed.

When it is determines that the injecting state of the liquid medicine LM is not abnormal (NO in S304), the control unit 16 ends the interruption process.

The interruption process shown in FIG. 6 is repeated at each predetermined time interval Δt during the operation of the micro-pump 12. The predetermined time interval Δt is determined to be smaller than an repletion interval of the processes from S206 through S214 shown in FIG. 5.

As described above in detail, according to the embodiment of the present invention, the liquid medicine injection amount adjusting apparatus 100 is connected in the middle of the liquid medicine injecting tube route formed from the container 10 which contains the liquid medicine LM to the biological body 22. The liquid medicine injection amount adjusting apparatus 100 includes the micro-pump 12, the flow volume sensor 14, and the control unit 16. The control unit 16 controls and monitors the power P of the micro-pump 12 so that the flow volume of the liquid medicine LM in the liquid medicine injecting tube route is maintained to be the target flow volume F₀ based on the flow volume F measured by the flow volume sensor 14. Therefore, even if the ambient temperature is changed, and/or the back pressure Pex to be operated against the micro-pump 12 from the biological body 22 (the blood vessel 23) is changed, the flow volume of the liquid medicine LM can be maintained to be the target flow volume F₀. In addition, the change of the back pressure Pex from the biological body 22 can be obtained from the monitored results of the power P of the micro-pump 12, and the injecting state of the liquid medicine LM can be obtained from the change of the back pressure Pex.

Consequently, abnormal injecting states such as the pulling out of a tip member in the liquid medicine injecting tube route, for example, the pulling out of the tip of the injection needle 20 from the biological body 22 can be immediately detected with high accuracy. In this case, the abnormal injecting state can be detected without disposing a sensor for obtaining the abnormal injecting state of the liquid medicine LM. Further, since the micro-pump 12 is used, the liquid medicine injection amount adjusting apparatus 100 can be realized with high usability in low cost and a small size.

In addition, according to the liquid medicine injecting system 200 of the embodiment of the present invention, since the liquid medicine injecting system 200 includes the liquid medicine injection amount adjusting apparatus 100, the liquid medicine injecting system 200 can immediately detect the occurrence of the above abnormal injecting state automatically with high accuracy. Therefore, continuation of the injection of the liquid medicine LM into the biological body 22 in the abnormal injecting state can be prevented.

In addition, according to the liquid medicine injecting system 200 of the embodiment of the present invention, since the injecting state of the liquid medicine LM is obtained by using the power P of the micro-pump 12 and/or the moving average of the time change rates of the power P, the liquid medicine injecting system 200 can stably operate the liquid medicine injection amount adjusting apparatus 100 without detecting a temporarily abnormal injecting state of the liquid medicine LM caused by the unstableness of the power source (the piezoelectric element 124) of the micro-pump 12, the unstableness of the feedback control, noise generated from measurement errors by the flow volume sensor 14, and a temporary change of the posture of the biological body 22.

In addition, when the posture of the biological body 22 is monitored, it can be determined whether an injecting state is a temporarily abnormal state based on the measured result of the posture of the biological body 22.

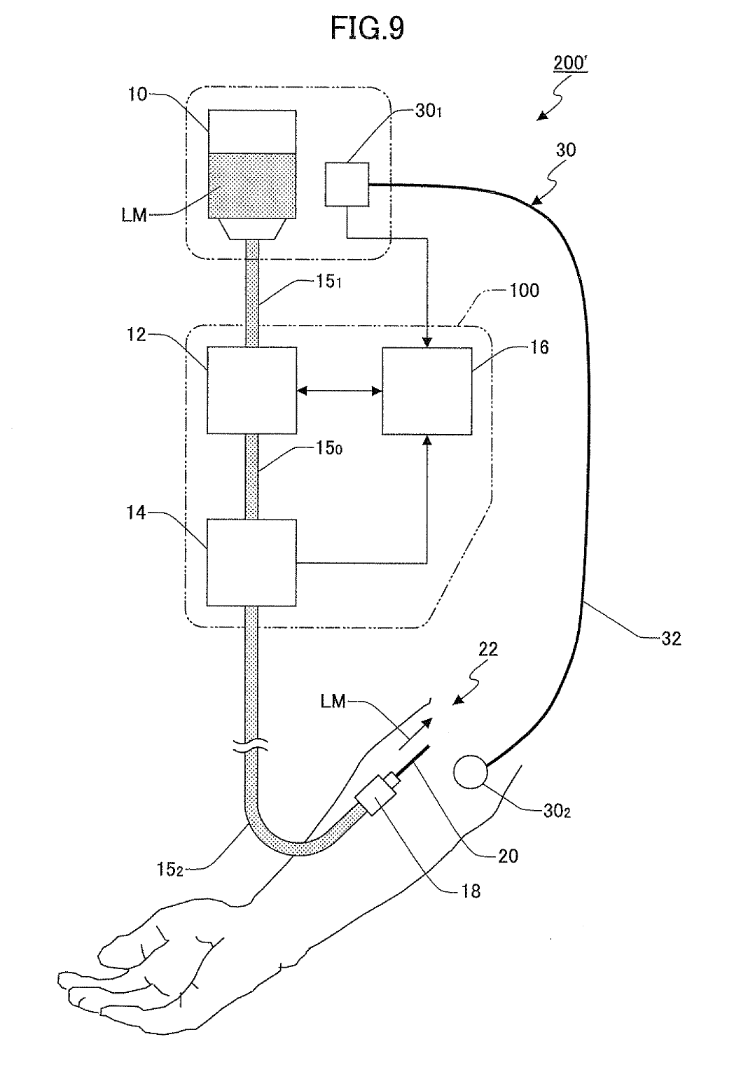

FIG. 9 is a diagram showing a structure of a liquid medicine injecting system 200′ according to a modified example of the embodiment of the present invention.

When the liquid medicine injecting system 200′ shown in FIG. 9 is compared with the liquid medicine injecting system 200 shown in FIG. 1, as shown in FIG. 9, the liquid medicine injecting system 200′ additionally includes a height measuring system 30 which measures a height difference between the container 10 which contains the liquid medicine LM and the injection needle 20 inserted into the blood vessel of the biological body 22.

The height measuring system 30 includes a main body 30₁ secured to the container 10 or positioned at the same height position of the container 10, an attaching pad 30₂ to be attached to the biological body 22, and a tube 32 which connects the main body 30₁ to the attaching pad 30₂. The inside of the tube 32 is filled with a liquid, for example, water, and a pressure sensor positioned in the attaching pad 30₂ which measures pressure from the liquid. The measures result by the pressure sensor is converted into a height difference between the main body 30₁ and the attaching pad 30₂ by the main body 30₁, and the converted result is sent to the control unit 16.

When the control unit 16 detects an abnormal injecting state of the liquid medicine LM by monitoring the power P described in the above embodiment, the control unit 16 obtains the measured result by the height measuring system 30. When a temporarily abnormal state occurs due to a change of the posture of the biological body 22, the control unit 16 can detect the abnormal injecting state from the monitored result of the power P and can simultaneously obtain the change of the posture of the biological body 22 from the measured result by the height measuring system 30.

When the control unit 16 obtains the change of the posture of the biological body 22 from the measured result by the height measuring system 30, the control unit 16 determines that the temporarily abnormal injecting state occurs. When the control unit 16 cannot obtain the change of the posture of the biological body 22 from the measured result by the height measuring system 30, the control unit 16 determines that the abnormal injecting state actually occurs. With this, even if the temporarily abnormal injecting state is detected by the change of the posture of the biological body 22, the liquid medicine injection amount adjusting apparatus 100 can be stably operated without stopping the injection of the liquid medicine LM into the biological body 22.

When the height of the injection needle 20 inserted into the blood vessel of the biological body 22 can be measured, the height measuring system 30 can be arbitrarily formed and the reference of the height in the height measuring system 30 can be arbitrarily determined.

In addition, when plural liquid medicines LM are simultaneously injected into the blood vessels of the biological body 22 by using two or more of the liquid medicine injecting systems 200′, one height measuring system 30 can be commonly used in two or more of the liquid medicine injecting systems 200′.

According to the embodiment of the present invention, as the flow volume sensor 14, the thermal type mass flow volume sensor is used. Therefore, the flow volume of the liquid medicine LM can be measured at high speed. Consequently, a high speed feedback control system can be realized in which the power P of the micro-pump 12 is immediately controlled corresponding to the change of the flow volume of the liquid medicine LM.

According to the embodiment of the present invention, as the micro-pump 12, a diaphragm pump (a kind of volume pumps) is used in which a driving source is the piezoelectric element 124. However, the micro-pump 12 is not limited to the diaphragm pump using the piezoelectric element 124. That is, the driving source is not limited to the piezoelectric element 124, and can be an electromagnet, a magnetostrictive element, and so on. In addition, the micro-pump 12 can be a volume pump other than the diaphragm pump. Since the micro-pump 12 is a volume pump whose driving source is the piezoelectric element 124, the micro-pump 12 can be used in a fluid having viscosity such as a compressible fluid, for example a gas, in addition to in a non-compressible fluid, for example, a liquid.

In addition, as the flow volume sensor 14, the thermal type mass flow volume sensor is used. However, when there is a sensor which can measure the flow volume without breaking the fluid, for example, an ultrasonic wave flow volume sensor can be used as the flow volume sensor 14. In addition, when a sensor can detect a flow volume of a fluid per unit time, or flow mass of a fluid per unit time, the sensor can be used as the flow volume sensor 14. Further, as the flow volume sensor 14, instead of using a sensor which directly measures the flow volume, a sensor can be used in which a flow rate is measure and the measured flow rate is converted into the flow volume.

In addition, in the embodiment of the present invention, the flow volume sensor 14 is positioned at the downstream side of the micro-pump 12, and the flow volume of the liquid medicine LM discharged from the micro-pump 12 is measured. However, the flow volume sensor 14 can be positioned at the upstream side of the micro-pump 12. In this case, the flow volume sensor 14 measures the flow volume of the liquid medicine LM to be supplied to the micro-pump 12.

In addition, in the embodiment of the present invention, the liquid medicine injection amount adjusting apparatus 100 individually includes the micro-pump 12 and the flow volume sensor 14 by connecting via the tube 15₀. However, the liquid medicine injection amount adjusting apparatus 100 can include one device in which the micro-pump 12 is integrated with the flow volume sensor 14 as one unit. In this case, the tube 15₀ is not required, and the liquid medicine injection amount adjusting apparatus 100 can be further small sized by having high usability. The device, in which the micro-pump 12 is integrated with the flow volume sensor 14 as one unit, can be manufactured by an MEMS technology.

In addition, in the embodiment of the present invention, as an example, the injecting state of the liquid medicine LM is determined by the interruption process. However, when the control unit 16 includes a high-speed CPU, the control unit 16 can determine the injecting state of the liquid medicine LM by using a so-called time sharing process.

In addition, in the embodiment of the present invention, as the biological body 22, a human body is implicitly assumed. However, the liquid medicine injection amount adjusting apparatus 100 and the liquid medicine injecting system 200 can be applied to an animal body. Further, the liquid medicine LM is injected into the blood vessel of the biological body 22. However, when a liquid medicine LM is required to be injected into an organ of the biological body 22, the liquid medicine injection amount adjusting apparatus 100 and the liquid medicine injecting system 200 can be used. Moreover, when a blood transfusion is required, the liquid medicine injection amount adjusting apparatus 100 and the liquid medicine injecting system 200 can be used.

### INDUSTRIAL APPLICABILITY

The liquid medicine injection amount adjusting apparatus 100 and the liquid medicine injecting system 200 according to the embodiment of the present invention can be suitably applied to a medical field when a liquid medicine LM is injected into a biological body 22.

Further, the present invention is not limited to the embodiment, but various variations and modifications may be made without departing from the scope of the present invention.

The present invention is based on Japanese Priority Patent Application No. 2008-205180 filed on Aug. 8, 2008, and Japanese Priority Patent Application No. 2009-154506 filed on Jun. 30, 2009 with the Japanese Patent Office, the entire contents of which are hereby incorporated herein by reference.

1. A liquid medicine injection amount adjusting method which adjusts an injection amount of a liquid medicine to be injected into a biological body from a container which contains the liquid medicine, comprising:
a first step which controls power of a pump connected in the middle of a liquid medicine injecting tube route formed from the container to the biological body so that a flow volume of the liquid medicine flowing in the liquid medicine injecting tube route is maintained to be a target flow volume based on measured information of the flow volume of the liquid medicine flowing in the liquid medicine injecting tube route; and
a second step which monitors the power of the pump in parallel with the first step.

2. The liquid medicine injection amount adjusting method as claimed in claim 1, wherein:
the second step further determines an injecting state of the liquid medicine into the biological body based on monitored information of the power of the pump.

3. The liquid medicine injection amount adjusting method as claimed in claim 2, wherein:
the pump is driven when a pulse voltage is applied to the pump; and
the second step measures a parameter relating to the power to be obtained from at least one of a pulse amplitude, a pulse width, and a pulse period of the voltage pulse which is applied to the pump, and monitors the power by using the measured result.

4. The liquid medicine injection amount adjusting method as claimed in claim 3, wherein:
when at least one of the measured result and a time change rate of the measure result is continuously more than a threshold value for a predetermined period, the second step determines that an abnormal injecting state occurs.

5. The liquid medicine injection amount adjusting method as claimed in claim 3, wherein:
the second step determines that an abnormal injecting state occurs based on a result in which a most recent time change rate of the time change rates of the measured results obtained at each predetermined time interval is compared with a threshold value.

6. The liquid medicine injection amount adjusting method as claimed in claim 4, wherein:
the second step obtains the time change rate by applying a least square method to the measured results.

7. The liquid medicine injection amount adjusting method as claimed in claim 4, wherein:
in a case where it is determined that the abnormal injecting state has occurred, the second step finally determines that the abnormal injecting state occurs when an average value per a predetermined time interval of at least one of the measured results and the time change rates of the measure results is more than a threshold value.

8. The liquid medicine injection amount adjusting method as claimed in claim 4, wherein:
the second step finally determines that the abnormal injecting state occurs when the number of determined occurrence times of the abnormal injecting state is more than the number of predetermined times.

9. The liquid medicine injection amount adjusting method as claimed in claim 2, wherein:
the second step further monitors a height of a tip of the liquid medicine injecting tube route at the side of the biological body, and determines the injecting state of the liquid medicine based on a monitored result.

10. The liquid medicine injection amount adjusting method as claimed in claim 9, wherein:
the second step measures a height difference between the container and the tip of the liquid medicine injecting tube route at the side of the biological body.

11. The liquid medicine injection amount adjusting method as claimed in claim 2, wherein:
when the abnormal injecting state of the liquid medicine is detected, the second step stops injecting the liquid medicine.

12. The liquid medicine injection amount adjusting method as claimed in claim 2, wherein:
when the abnormal injecting state of the liquid medicine is detected, the second step gives a warning.

13. The liquid medicine injection amount adjusting method as claimed in claim 1, wherein:
the first step stops injecting the liquid medicine into the biological body when the target amount of the liquid medicine has been injected into the biological body.

14. A liquid medicine injection amount adjusting apparatus which is connected in the middle of a liquid medicine injecting tube route formed from a container which contains a liquid medicine to a biological body into which the liquid medicine is injected and adjusts an injection amount of the liquid medicine into the biological body, comprising:
a pump positioned at a position in the middle of the liquid medicine injecting tube route for running the liquid medicine flowing in the liquid medicine injecting tube route;
a measuring unit positioned at another position in the middle of the liquid medicine injecting tube route which measures a flow volume of the liquid medicine flowing in the liquid medicine injecting tube route; and
a control unit which controls power of the pump based on a result measured by the measuring unit so that the flow volume of the liquid medicine is maintained to be a target volume and monitors the power of the pump.

15. A liquid medicine injecting system, comprising:
the liquid medicine injection amount adjusting apparatus as claimed in claim 14.
